(19) United States
(12) Patent Application Publication
(10) Pub. No.: US 2006/0100658 A1
(43) Pub. Date: May 11, 2006

(54) Interventional guiding sheath system and method of use

(76) Inventors: Hiroyuki Obana, Wilmington, DE (US); Fumihisa Hirose, Tokyo (JP); James P. Rushworth, Doylestown, PA (US)

Correspondence Address: CERMAK & KENEALY, LLP, 515 EAST BRADDOCK RD SUITE B, Alexandria, VA, 22314, US

(21) Appl. No.: 10/983,703

(22) Filed: Nov. 9, 2004

(51) Int. Cl. A61M 29/00

(52) U.S. Cl. 606/200

(57) ABSTRACT

An interventional guiding sheath system can include an inner sheath, an outer sheath, and a filter located between the inner sheath and outer sheath. The filter can be expandable such that it opens to secure the guiding sheath system within a vessel of a patient during an interventional procedure. The inner sheath can include a mesh or other type of opening such that when the filter is collapsed back against the inner sheath, any debris collected by the filter during the procedure can pass through the inner sheath for aspiration out of the vessel. Additionally, a dilator can be used with the inner sheath for, among other things, aiding in placement of the device. The dilator can also be provided with a mesh, a recess, or other type of opening to allow any debris that is collected by the filter to either be stored by the dilator or aspirated from the dilator.

### BACKGROUND OF THE INVENTION

1. Field of the Invention

The present invention relates to an interventional device for protecting a patient from embolization and other problems during interventional procedures, and for stabilizing medical devices during vascular interventional procedures. More specifically, this invention relates to a guiding sheath system that provides a filter downstream of an intervention/surgery site such that a patient is protected from embolization and such that the guiding sheath system is stabilized, locked and/or centered during the interventional procedure.

2. Description of the Related Art

A variety of interventional procedures (both surgical and non-surgical), including angioplasty, artherectomy, stenting and the like have been developed for removing debris and/or obstructions from blood vessels. For example, balloon angioplasty utilizes a balloon-tipped catheter which is inserted in a stenosed or otherwise damaged or blocked region of a blood vessel. The stenosed, blocked or damaged region of the blood vessel is dilated by locating the balloon-tipped catheter at an appropriate location and inflating the balloon. Different types of vascular surgery can include either removing plaque or other obstruction from the artery or attaching a graft to the artery so as to bypass the obstruction. Other techniques, such as artherectomy, have also been proposed. In artherectomy, a rotating blade is used to shave plaque from an arterial wall.

One problem common with these techniques is the accidental release of portions of plaque, tissue, or thrombus, resulting in possible emboli which can lodge elsewhere in the vascular system. Such emboli can result in embolisms or other dangerous impairments of the circulatory system, such as a stroke, myocardial infarction or limb ischemia, etc.

Vascular filters or embolism traps have previously been proposed and are often used during a postoperative period, when there is a perceived risk of a patient encountering a pulmonary embolus resulting from clots, debris, etc., generated at the interventional site. In a typical use of vascular filters, the filter is mounted in the vena cava to catch large emboli passing from the intervention/surgery site to the lungs. These types of vascular filters are usually permanently implanted in the venous system of the patient, so that even after the need for the filter has abated, the filter remains in place for the lifetime of the patient.

Recently, removable/non-permanent vascular filters have been developed for use in artherectomy procedures. For example, U.S. Pat. No. 6,682,543 discloses methods for aortic artherectomy in which a guiding catheter is used to position and stabilize an artherectomy catheter within the vessel of a patient. The guiding catheter may carry a filtration assembly and allow passage of the artherectomy catheter through a lumen of the guiding catheter. In use, the guiding catheter will be positioned and its filter deployed. At the end of the procedure, the filtration device will be contracted to reduce its size, and will eventually be removed along with the guiding catheter from the patient. Notably, all plaque material excised during this procedure remains trapped either within the catheter housing under suction, or within the filtration mesh once collapsed. In this manner, U.S. Pat. No. 6,682,543 describes a method for aortic artherectomy including how to protect a patient from embolization during an aortic artherectomy procedure.

Another type of filter that is used during vascular intervention and that is commonly known in the art is a filter-type distal protection device. In such a device, a filter can be located downstream of and distal to an interventional procedure site to prevent any debris from moving distally downstream of the site.

It would be desirable to provide a non-permanent/removable filter device which could be located within the vascular system to more efficiently collect and retrieve debris, plaque, thrombus and other emboli which have dislodged during surgery, angioplasty, artherectomy or other interventional procedure. In addition, it would be desirable to have a filter that can be located downstream of the surgical/interventional device and which can be incorporated into or used in conjunction with a guiding sheath such that it can be easily incorporated into the interventional procedure with a minimum of additional steps. For example, it is contemplated that a filter could be connected to an inner sheath and collapsible onto a mesh, holes, or other debris collecting portion on the inner sheath to facilitate aspiration or removal of debris.

Furthermore, it would be beneficial for a filter that is deployable from a guiding sheath to also serve the function of stabilizing and centering the guiding sheath(s) or catheters for stability during the interventional procedure. The filter could prevent reactionary movement of the guiding sheath system due to reaction forces resulting from the interventional procedure.

There also remains a need for more efficient removal of debris that travels downstream of an interventional site. Therefore, it is contemplated that providing a filter that acts in conjunction with a guide sheath that has a mesh portion, plurality of holes portion, slit portion or other means for capturing and/or aspirating debris would be beneficial. Further, it is believed that a dilator could be provided that includes means for aspirating or removing debris such as a mesh portion, holes portion, slit portion, indent, a sealed gap that is formed by a tip occluder, etc. In particular, the sealed gap can be defined by the dilator wall, the inner sheath wall and the tip occluder. The sealed gap would provide an area for debris to be aspirated into or simply stored in after a portion of the interventional procedure is complete. For example, a dilator with a tip occluder can be inserted into the inner sheath after a medical procedure on a vessel is finished. The inner sheath, filter, and dilator can all be withdrawn into the outer sheath to cause debris trapped in the filter to pass through the inner sheath and into the sealed gap defined by the dilator, tip occluder and inner sheath. The debris can be removed by aspiration or by simply removing the entire guide sheath system. Thus, the dilator could act in conjunction with the inner sheath and filter to facilitate removal of debris.

### SUMMARY OF THE INVENTION

In accordance with one of several aspects of the present invention, a guiding sheath system for use in an interventional procedure on a patient can include an inner sheath configured to guide an interventional device to a region of interest in the patient, an outer sheath configured to be slidable along the inner sheath, and a filter located adjacent the inner sheath and configured to be expandable.

In accordance with another aspect of the invention, a guiding sheath system for use in an interventional procedure can include an inner sheath including a debris portion, sheath located adjacent the inner sheath, and a filter capable of expanding between a contracted state and an expanded state. The filter can be configured such that it can be located adjacent the debris filter portion of the inner sheath when in the contracted state during use.

In accordance with another aspect of the invention, the debris portion of the inner sheath can be formed as one of a mesh portion, a slit portion, and a plurality of holes portion.

In accordance with yet another aspect of the invention, the dilator can be located adjacent the inner sheath. The dilator can also include one of an indent that forms a gap between the dilator and inner sheath, a plurality of holes portion, a slit portion, and a mesh portion.

In accordance with still another aspect of the invention, a method for protecting a patent from embolization, and more particularly distal embolisms, resulting from an interventional procedure can include providing a guiding sheath system that includes an inner sheath, an outer sheath, and a filter located adjacent the inner sheath, advancing the guiding sheath system to a region of interest in a vessel of the patient, expanding the filter at a location downstream of the region of interest, deploying a medical instrument through the guiding sheath, performing an interventional procedure with the medical instrument on the patient, and capturing material dislodged by the procedure with the filter.

Additional features, advantages, and embodiments of the invention may be set forth or apparent from consideration of the following detailed description, drawings, and claims. Moreover, it is to be understood that both the foregoing summary of the invention and the following detailed description are exemplary and intended to provide further explanation without limiting the scope of the invention as claimed.

### DESCRIPTION OF THE DRAWINGS

The above and other aspects, features and advantages of the present invention will become more apparent from the following detailed description and reference to preferred embodiments of the apparatus and method, given only by way of example, when taken in conjunction with the accompanying drawings, in which:

FIG. 1 is a partial longitudinal view of a guiding sheath system made in accordance with the principles of the invention with a filter located in the aorta downstream of the renal arteries;

FIG. 2 is a partial sectional view of a guiding sheath system made in accordance with the principles of the invention;

FIG. 3 is a partial sectional view of another embodiment of a guiding sheath system made in accordance with the principles of the invention;

FIG. 4 is a partial sectional view of another embodiment of a guiding sheath system made in accordance with the principles of the invention;

FIG. 5 is a front elevational view of another embodiment of a guiding sheath system and operational structures made in accordance with the principles of the invention;

FIGS. 6(a)-6(c) are front elevational views of the guiding sheath system and operational structures of FIG. 5 showing a chronology of operational steps;

FIGS. 7(a)-7(g) are partial cross-sectional views of a guiding sheath system made in accordance with the principles of the invention and showing a chronology of operational steps;

FIG. 8 is a partial front elevational view of a guiding sheath system that is made in accordance with the principles of the invention and that includes a shaped inner sheath located in an aorta;

FIG. 9 is a partial front elevational view of a guiding sheath system that is made in accordance with the principles of the invention and that includes a filter type distal protection device;

FIG. 10 is a partial front elevational view of a guiding sheath system that is made in accordance with the principles of the invention and that includes a catheter located in an aorta; and

FIG. 11 is a Table showing twelve (12) exemplary combinations of guiding sheath system structures arranged in accordance with the principles of the invention.

### DETAILED DESCRIPTION OF PREFERRED EMBODIMENTS

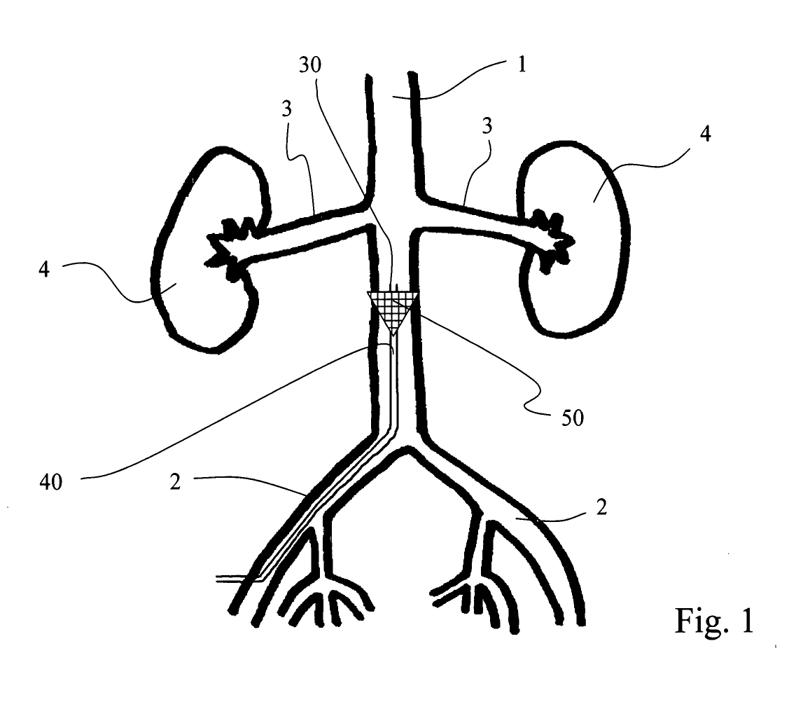

Preferred embodiments of the present invention will be described below in detail with reference to FIGS. 1 to 11. Referring to the drawing figures, like reference numerals designate identical or corresponding elements throughout the several figures. It is to be noted that while the embodiments described below are preferred specific examples of the present invention and therefore have various technically preferred features, the present invention is not limited thereto.

FIG. 1 shows a partial view of an embodiment of a guiding sheath system with a filter 50 that is placed near the renal arteries 3. The filter 50 is preferably expanded in the aorta 1 downstream of the renal arteries 3 and kidneys 4. The filter 50 can be configured such that when it expands, it locks onto the walls of the aorta 1 to secure the guiding sheath system at approximately the center of the aorta 1. Thus, the interventional device and/or the guiding sheath can be prevented from flexing, for example backing off, when inserted through the inner sheath 30 such that it is not biased towards a wall of the vessel or artery as the tip of the interventional device is being moved towards a surgical/intervention site, resulting in proper control of movements of the distal end of the interventional device as the operator desires. In addition, the filter 50 can be configured to help prevent the guiding sheath and/or interventional device from touching or hurting the wall of the aorta or other vessel. The filter 50 can also prevent the guiding sheath system from moving backward (i.e., in a downstream downward direction in FIG. 1) when the components of the guiding sheath system or other interventional components are advanced towards a region of interest in a patient. Thus, the filter 50 counteracts the reactionary forces during operation/extension of components in or of the guiding sheath. The placement of the filter 50 also allows any emboli, plaque, tissue or other debris to be captured by the filter before they flow downstream to the femoral arteries 2.

The guiding sheath system of FIG. 1 is shown positioned for interventional procedures on the aorta, renal arteries or kidneys. Typically, during interventional procedures on the renal arteries, an incision is made in one of the femoral arteries and the guiding sheath is inserted through the femoral artery. The guiding sheath travels along the femoral artery and subsequently travels along the aorta to a location just below the renal arteries. (The inventive methods for using the guiding sheath system are discussed in greater detail below). When the guiding sheath system has reached its intended position below the renal arteries, the filter 50 can be expanded to contact the aorta wall and lock the guiding sheath system in place, as shown in FIG. 1.

The guiding sheath system of FIG. 1 is optimally placed for interventional procedures on the renal arteries. However, the guiding sheath system of the invention can be placed in various other locations depending on the interventional procedure being conducted with the guiding sheath system. For example, the filter can be placed in the aorta or arteries adjacent the aorta, such as just below (downstream of) the celiac trunk artery for conducting interventional procedures on the common hepatic artery or the aorta In addition, it is contemplated that the invention can be used in many other areas of the vascular system, including but not limited to the common, external, and internal carotid arteries, the basilar artery, the brachiocephalic trunk, the middle, anterior, and posterior cerebral arteries, the vertebral artery, the lumbar artery, the hepatic artery, the subclavian artery, the brachial artery, the axillary artery, the iliac artery, the renal artery, the femoral arteries, the popliteal artery, the celiac artery, the superior and inferior mesenteric arteries, the anterior and posterior tibial arteries and all other arteries carrying oxygenated blood. The invention can also be used in the venous system of a patient.

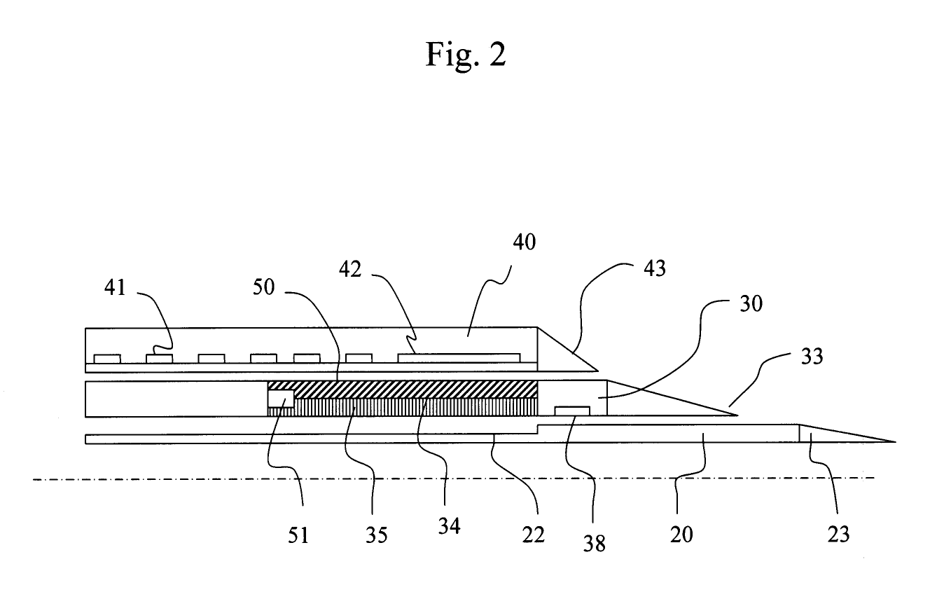

FIG. 2 shows a partial cross-section view of a guiding sheath system made in accordance with the principles of the invention. The guiding sheath system can include an outer sheath 40 that is preferably reinforced by coil or braided reinforcements 41 preferably made of stainless steel or the like. A radiopaque marker 42 can be located at a distal end of the outer sheath 40. The tip 43 of the outer sheath 40 is preferably tapered sufficiently to facilitate smooth insertion of the guiding sheath system at the incision and prevent damage of a blood vessel wall during operation of the guiding sheath system.

An inner sheath 30 can be located in the outer sheath 40. A radiopaque marker 38 can be located at a distal end of the inner sheath 30 so that the position of the inner sheath 30 can be easily determined during the interventional procedure. The tip 33 of the inner sheath 30 is preferably tapered sufficiently to facilitate smooth insertion of the guiding sheath system at the incision and prevent damage of a blood vessel wall during operation of the guiding sheath system. The inner sheath 30 as shown in FIG. 2 can include a filter 50 located directly adjacent and in contact with an inner sheath debris portion 34. The filter 50 can be connected to the inner sheath debris portion 34 and/or inner sheath 30 by a bond 51. The filter 50 can also be self expandable such that when the outer sheath 40 is not located over the inner sheath 30, the filter 50 expands to contact the walls of the blood vessel and thereby trap debris traveling downstream in the aorta (or other vessel) from an interventional site. After an interventional procedure the outer sheath 40 can be slid back over the inner sheath 30 and the filter 50 can be contracted onto the debris portion 34 of the inner sheath 30. The debris portion 34 can include small holes 35 configured to permit debris to pass therethrough or be caught therein for aspiration or other removal from a patient. Thus, debris caught in the filter 50 can be contracted or aspirated through the small holes 35 in the inner sheath 30.

A dilator 20 can also be provided with the guiding sheath system. The dilator 20 can be located in and can be slidable relative to the inner sheath 30. In the embodiment of FIG. 2, the dilator 20 includes a dilator indent 22 that is configured to retain emboli or other debris therein for removal during or after an interventional procedure. The dilator indent 22 can be located immediately adjacent the inner sheath debris portion 34 such that debris that pass through the small holes 35 will be trapped in the dilator indent 22 for removal. The dilator 20 can also include a dilator tip 23 that is preferably tapered sufficiently to facilitate smooth insertion of the guiding sheath system at the incision and prevent damage of a blood vessel wall during operation of the guiding sheath system.

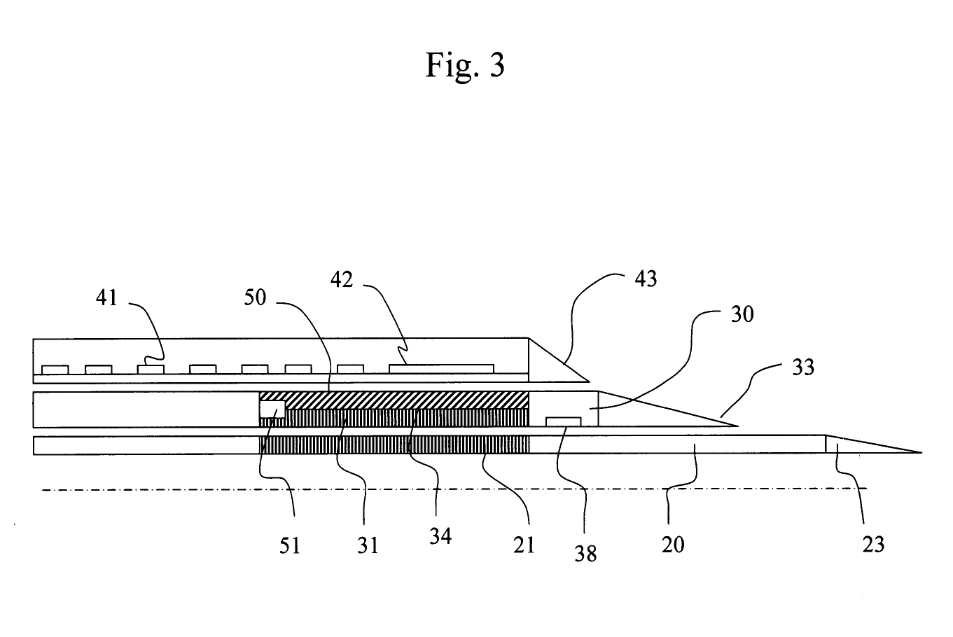

FIG. 3 shows a partial cross section of another embodiment of a guiding sheath system made in accordance with the principles of the present invention. In this embodiment, the dilator 20 can include a dilator mesh portion 21. The dilator mesh portion 21 can be configured such that it can be immediately adjacent the inner sheath debris portion 34 at a particular time during an interventional procedure. The debris portion 34 of the inner sheath 30 can be configured as a mesh portion 31, as shown in FIG. 3. Thus, debris can pass through both dilator mesh portion 21 and inner sheath mesh portion 31 for aspiration or other removal from the patient.

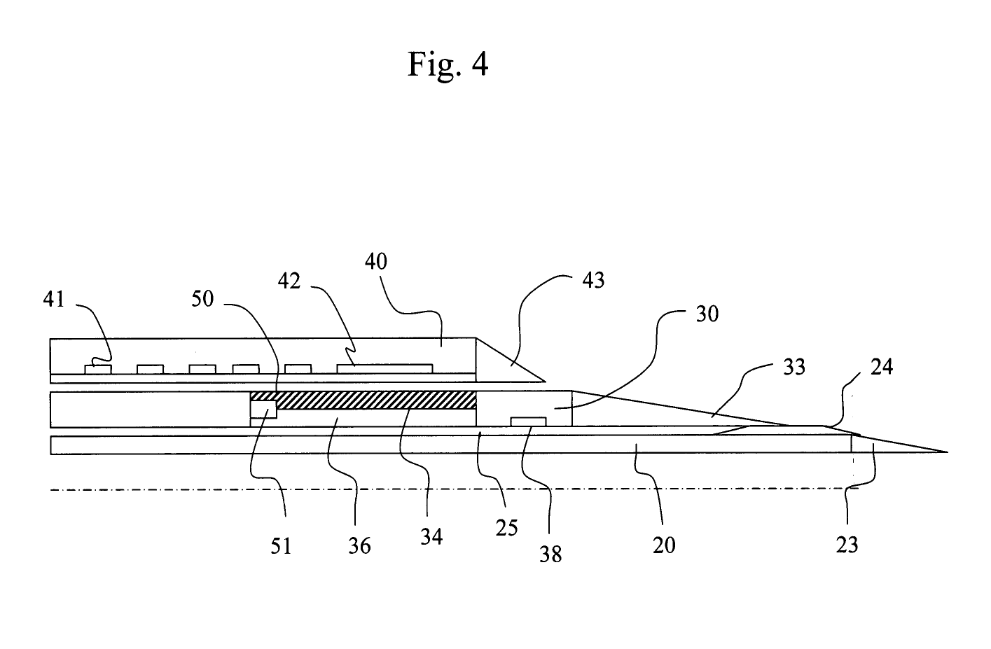

FIG. 4 shows a partial cross section of another embodiment of a guiding sheath system made in accordance with the principles of the invention. In this embodiment, the dilator 20 can include a tip occluder 24 located adjacent the dilator tip 23. The tip occluder 24 can be configured to directly contact the inner circumference of the sheath 30 such that the tip occluder 24 forms a gap 25 between the dilator 20 and inner sheath 30. The tip occluder 24 can thus segregate the gap 25 located between the inner sheath 30 and dilator 20 from the space located outside of the distal end of the inner sheath 30 and dilator 20 (e.g., the blood vessel or interventional area). Accordingly, any emboli or other debris that become trapped in the gap 25 located between the dilator 20 and inner sheath 30 can be aspirated or otherwise removed during or after the interventional procedure, thus preventing the emboli or other debris from moving from the gap 25 to the space exterior of the distal end of the dilator 20 and inner sheath 30. The debris portion 34 is configured as a slit 36 through which debris can be trapped or travel through for aspiration or removal from the guiding sheath system.

The debris portion 34 in any of the disclosed embodiments can alternatively be configured as a mesh portion, small holes portion, slit portion, permeable membrane portion, indent or other structure or area for trapping, aspirating or otherwise removing debris. Those of skill in the art will readily understand the structure and use of these alternatives, and therefore further details thereof are not included so as not to obscure the principles of the present invention.

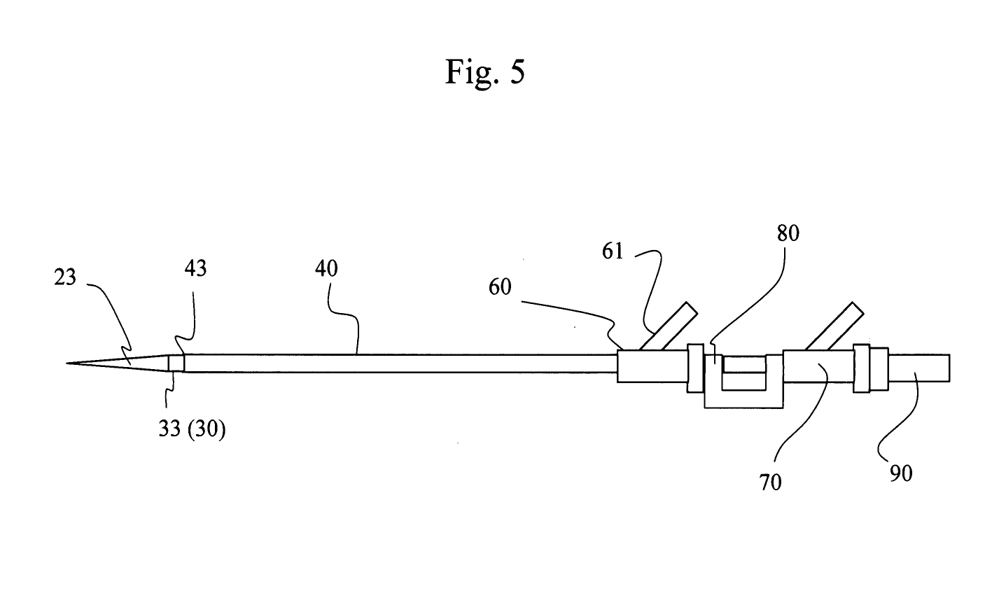

FIG. 5 depicts an embodiment of a guiding sheath system and its operational structures made in accordance with the principles of the invention. The guiding sheath system of FIG. 5 can include a dilator 20 that, when positioned within the outer sheath 40, has a dilator tip 23 extending from the inner sheath 30. A sheath hub 60 can be located at a proximal region of the outer sheath 40. A side port 61 can be used to inject saline to prime the gap between the outer sheath 40 and inner sheath 30. The sheath hub 60 can be formed as a modified version of a Tuohy-Borst valve 70, or other similar valve.

A spacer 80 can be provided adjacent the sheath hub 60 to separate the sheath hub 60 from a Tuohy-Borst valve 70 or the like, and a dilator hub 90. Accordingly, when the guiding sheath system is inserted in a patient, the spacer 80 can be removed to permit retraction of the outer sheath 40 and/or other structures relative to the dilator 20 and Tuohy-Borst Valve 70. The dilator hub 90 can be used to withdraw the dilator 20 from the inner sheath 30 and outer sheath 40 when necessary during an interventional procedure.

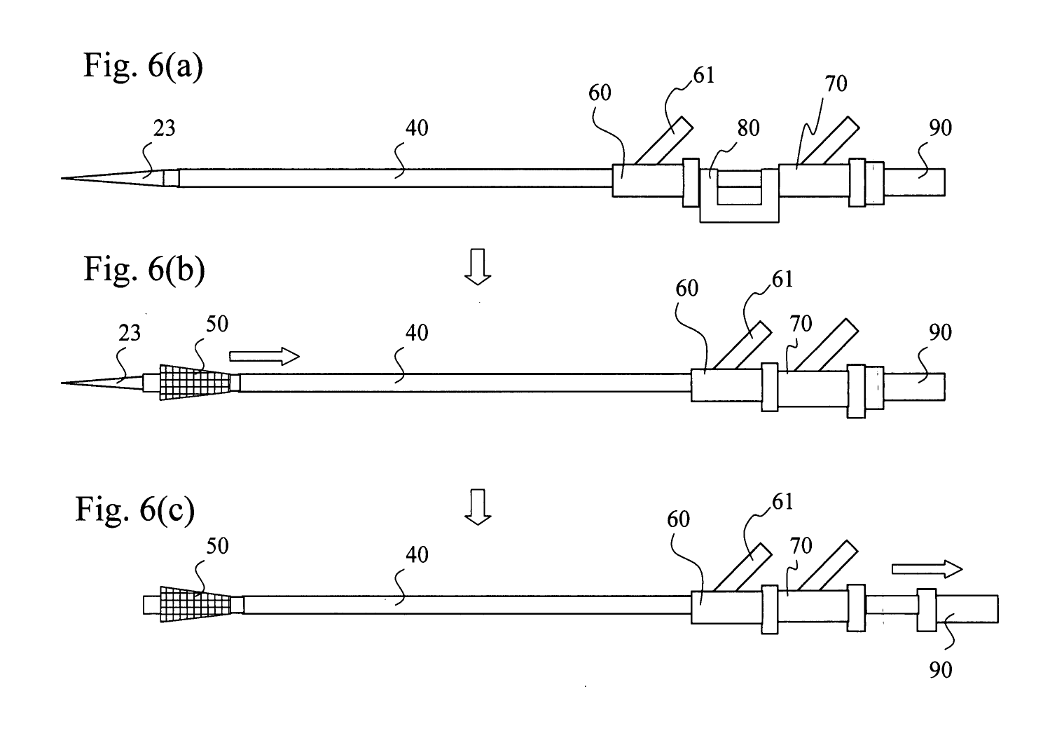

FIGS. 6(a)-6(c) depict a series of possible operational steps for the guiding sheath system as shown in FIG. 5. FIG. 6a depicts the guiding sheath system in its initial state. The sheath hub 60 is located immediately adjacent the spacer 80, which in turn is located immediately adjacent the Tuohy-Borst valve 70, which in turn is immediately adjacent the dilator hub 90. In this state, the guiding sheath system can be inserted into a patient's blood vessel, such as the femoral artery. The guiding sheath system can then be advanced and located at a region of interest in a patient (e.g., just below/downstream of the renal arteries) by conventional interventional techniques. The location can be specifically determined by viewing the radiopaque marker 42 via fluoroscopic video or the like.

Once the guiding sheath system is located at a region of interest in a patient, the spacer 80 can be removed and the sheath hub 60 can be retracted along the inner sheath 30, as shown in FIG. 6(b). This motion retracts the outer sheath 40 from the inner sheath, exposing a filter 50. The filter 50 can be configured to expand when uncovered by the outer sheath 40. The filter 50 preferably expands to contact the vessel walls (e.g. aorta wall) to lock the inner sheath 30 and filter 50 in place within the patient. As shown in FIG. 6(c), once the filter 50 is expanded in place in the patient, the dilator 20 can be partially withdrawn or totally removed from the inner sheath 30 by moving the dilator hub 90 relative to the sheath hub 60 and Tuohy-Borst valve 70.

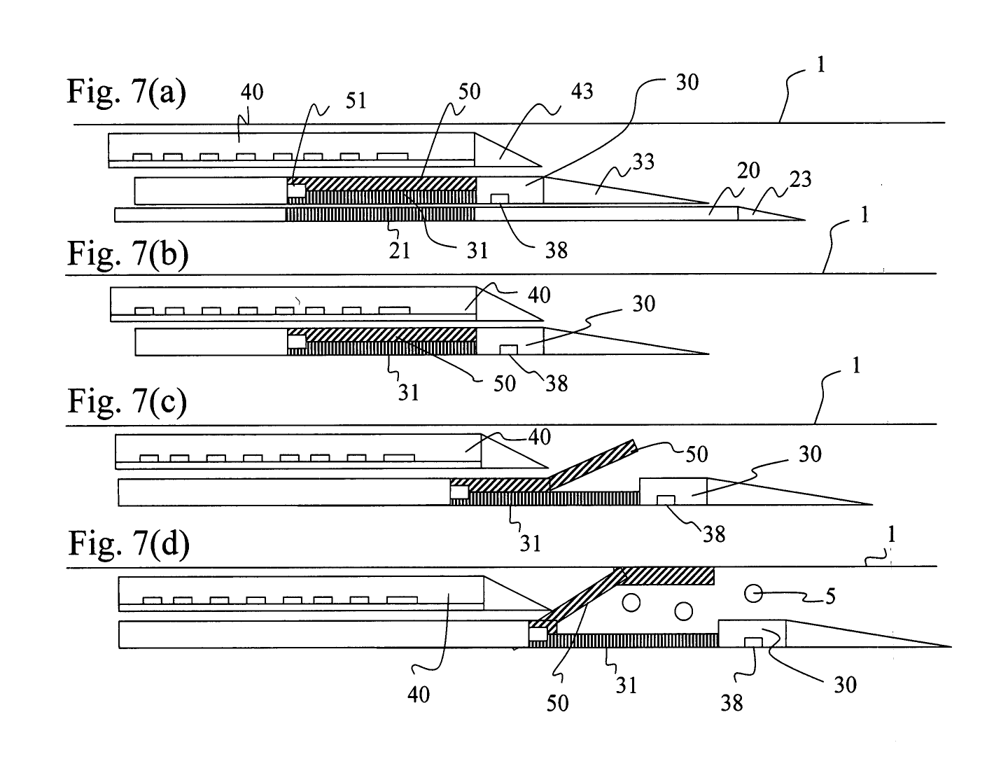

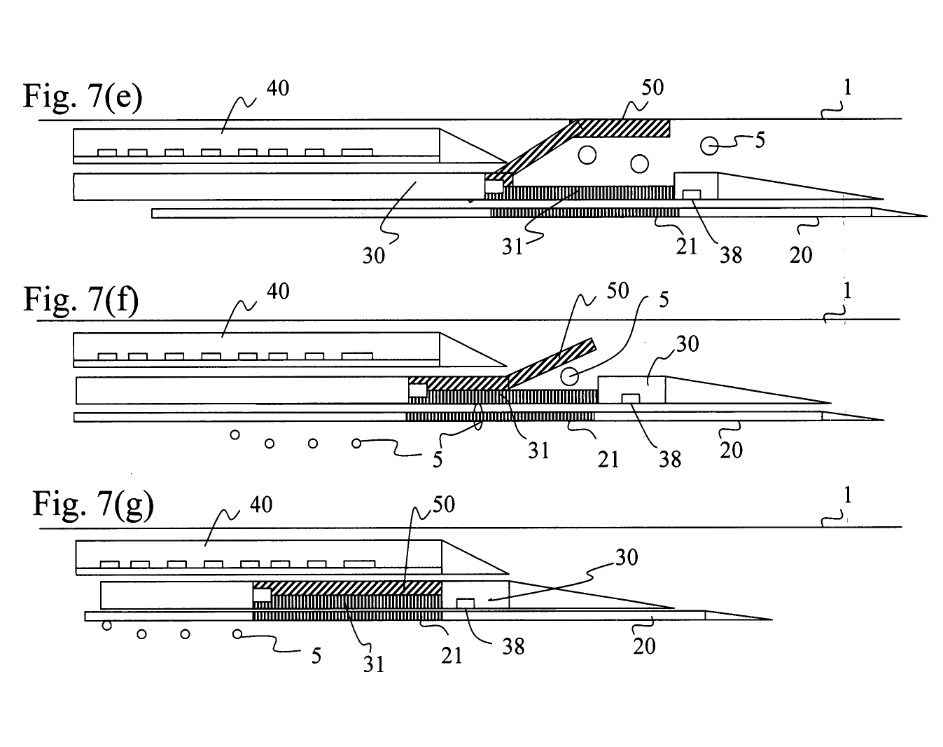

FIGS. 7(a)-7(g) show a partial cross-section of a guiding sheath system and depict contemplated chronological steps of using the guiding sheath system. As shown in FIG. 7(a), the guiding sheath system can include a dilator 20, an inner sheath 30 and an outer sheath 40. The dilator tip 23 extends from the inner sheath tip 33 which in turn extends from the outer sheath tip 43 in FIG. 7(a) such that the guiding sheath system is oriented for initial positioning within a patient. The interventional procedure can start with a surgeon or other medical personnel making an incision in one of the femoral arteries 2 and then inserting a cannula into the femoral artery 2, e.g., using a Seldinger or cut-down technique. An access guidewire (typically 0.035″ or 0.038″) can then be inserted into the femoral artery 2 via the cannula. Once the access guidewire is inserted, the cannula can be removed from the guidewire.

Next, the dilator 20, inner sheath 30, and outer sheath 40 can be inserted over the access guidewire into the femoral artery 2. In this embodiment, the dilator 20 can have a mesh portion 21 that is configured to generally coincide in position with the mesh portion 31 of the inner sheath 30 when both are in the extended initial position. In this position, the inner sheath 30, dilator 20 and outer sheath 40 can be advanced through the femoral artery and then through the aorta to a position just below the renal arteries (when conducting a procedure, for example, on a renal artery). Once in position, both the dilator 20 and access guide wire can be removed from the inner sheath 30, as shown in FIG. 7(b).

The outer sheath 40 can then be withdrawn and moved towards the proximal end of the guiding sheath system to uncover the filter 50. As shown in FIG. 7(c), the filter 50 can be configured to self-expand when it is uncovered by the outer sheath 40. FIG. 7(d) shows the guiding sheath system in its fixed operational mode during an interventional procedure. Specifically, during an interventional procedure, the filter 50 can be locked to the wall of the aorta 1 to thereby secure the inner sheath 30 with respect to the aorta 1. Thus, instruments can be inserted to a region of interest in a patient via the inner sheath 30 without any reactionary motion of the guiding sheath system due to reactive forces occurring during the interventional procedure.

During the interventional procedure, any debris 5 such as loose tissue, plaque fragments or other emboli can be collected by the filter 50 when the guiding sheath system is in the position shown in FIG. 7(d). The filter can be configured to trap any debris 5 that moves downstream in the aorta 1, thus preventing the debris from moving downstream and into the femoral arteries where they can possibly cause an embolism or other vascular problem.

During the interventional procedure, an interventional guide wire (usually 0.018″ or 0.014″) is typically inserted through the inner sheath 30 when the guiding sheath system is configured as shown in FIG. 7(d). An interventional device such as an angioplasty balloon catheter or stent system can then be placed over the interventional guide wire and advanced through the inner sheath 30 until it reaches a region of interest in the patient, such as a lesion, deformity or injury in an artery. The interventional procedure is then conducted on the region of interest. During this procedure, any debris 5 that becomes dislodged is captured by the filter 50 located downstream of the region of interest.

Once the interventional procedure is completed, the interventional device can be removed from over the interventional guide wire, and the interventional guide wire can also be removed from the inner sheath 30. The dilator 20 can then be re-inserted into the inner sheath 30 such that the dilator mesh portion 21 coincides in position with the inner sheath mesh portion 31, as shown in FIG. 7(e).

Next, the outer sheath 40 can be slid along the inner sheath 30 to cause the filter 50 to retract into its original position between the outer sheath 40 and inner sheath 30. As the outer sheath 40 causes the filter 50 to retract, any debris 5 that is caught in the filter is moved towards and compressed into the inner sheath mesh portion 31, as shown in FIG. 7(f). The debris 5 can eventually be compressed either into or through the inner sheath mesh portion 31 and then subsequently into or through the dilator mesh portion 21. The outer sheath 40 can be slid to fully encapsulate the filter 50, thus completely compressing the filter 50 against the inner sheath 30 and/or inner sheath mesh portion 31.

The mesh or apertures of the inner sheath mesh portion 31 and dilator mesh portion are preferably rougher/larger than the mesh or apertures of the filter 50 to facilitate passage of the debris 5 from the filter through the various passways of the guiding sheath system. Debris 5 that is compressed through both the inner sheath mesh portion 31 and dilator mesh portion 21 can be aspirated from the inner lumen of the dilator 20 by conventional methods of aspiration, as shown in FIG. 7(g). Any debris 5 that remains in the filter 50, inner sheath mesh portion 31, or dilator mesh portion 21 can be removed by removal of these same parts from the patient.

If the dilator 20 includes a dilator indent 22 (see FIG. 2, for example) instead of a dilator mesh portion 21 (see FIG. 3, for example), the debris that passes through the inner sheath mesh portion 31 can be trapped within or aspirated from the dilator indent 22 when the dilator 20 is inserted into the inner sheath 30. It should be noted that the dilator 20 can be inserted in the inner sheath 30 before a specific portion of an interventional procedure to help guide the guiding sheath system to a location within a vessel (for example, either during assembly of the guiding sheath system or during a set-up portion of the interventional procedure). Once the guiding sheath system is located in the vicinity of interest in a patient's vessel, the dilator 20 can be removed from the inner sheath 30. A medical device can be inserted into the inner sheath 30 and a specific procedure can then be performed on the patient's vessel. After that specific procedure is performed, the medical device used during the procedure can be removed from the inner sheath 30. The dilator 20 can then be reinserted into the inner sheath 30, forming a gap defined between the dilator indent 22 and inner sheath 30. The filter 50 can be withdrawn back into the outer sheath when the dilator 20 is reinserted in the inner sheath 20. Any debris located in the filter 50 can then pass through the inner sheath mesh portion 31 and into the dilator indent 22. The dilator 20 can thus facilitate movement or withdrawal of the inner sheath 30 and filter 50 into the outer sheath 40, and can also facilitate removal of the guiding sheath system in general. As indicated above, the dilator indent 22 can trap or allow aspiration of debris that passes through the inner sheath 30 and into the dilator indent 22. No aspiration is required when this embodiment of the guiding sheath system is used in conjunction with an interventional procedure. In addition, no aspiration of the central dilator lumen is required when a dilator 20 that includes a tip occluder 24 (see FIG. 4, for example) is used with the interventional procedure. Moreover, debris 5 can be trapped between the dilator 20 and the inner sheath 30 when a tip occluder 24 is present on the dilator 20.

When using an embodiment of the guiding sheath system that includes a dilator indent 22 or dilator tip occluder 24, aspiration of the area between the dilator 20 and the inner sheath 30 can be undertaken to remove the debris, if desired.

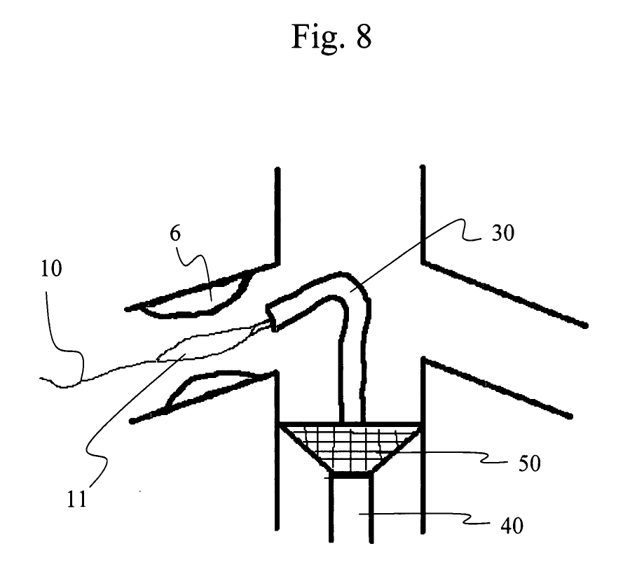

FIG. 8 shows an embodiment of the guiding sheath system in which the inner sheath 30 is formed as a long tip and is shaped such that it can extend from the filter 50 and take up a position adjacent a lesion 6 in a patient. A guidewire 10 can be fed through the inner sheath 30 to the lesion area 6 in the patient. If the interventional procedure includes some type of angioplasty procedure, a balloon catheter 11 can be guided via the guidewire 10 to the lesion 6 and typical angioplasty and/or stenting procedures can be undertaken.

The inner sheath 30, outer sheath 40, guidewires, and catheters used with the guiding sheath system can be shaped by including shape oriented materials or other known materials or structures for providing a shaped construction. For example, a shape memory alloy portion can be located on the inner sheath 30 such that when the inner sheath 30 is extended into a vessel, the inner sheath 30 warms to body temperature, and the shape memory alloy causes the inner sheath to take on a predetermined shape for facilitating passage of the inner sheath 30 through the particular geometry of the patient's vessel. In addition, the inner sheath 30 can be shaped by being bent or loaded for predisposition to a particular shape, either by force, by heat or by other choice of materials. A shaped inner sheath 30 formed by loading can take a predetermined position or shape when it is removed from the outer sheath 40 by the elastic nature of the inner sheath 30 itself. Whether to use a shaped or a non-shaped structure for a component of the guiding sheath system can be determined by many different factors, including the patient's anatomy, the type of interventional procedure, the preference of the medical personnel, etc.

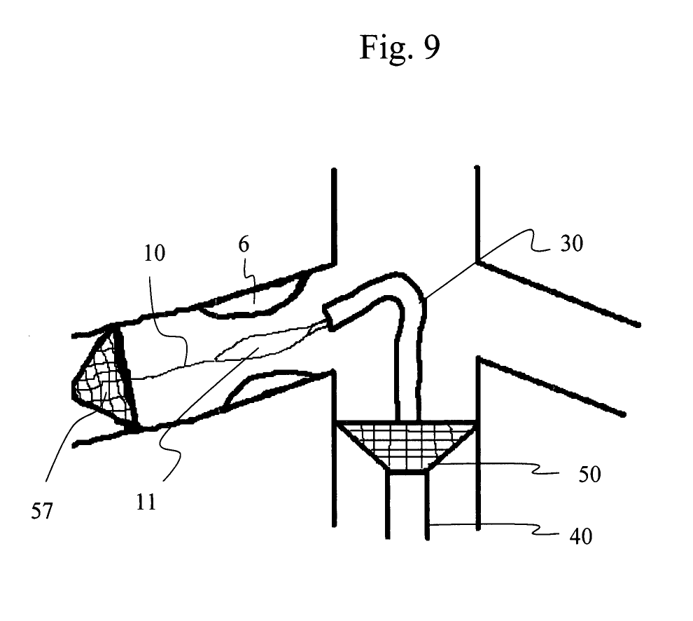

FIG. 9 shows another embodiment of the guiding sheath system in which a distal protection device such as a filter-type distal protection device 57 is located downstream of the damaged, diseased or otherwise problematic area (target area) of a vessel. The filter-type distal protection device 57 can be placed opposite to the filter 50 with relation to the target area of the vessel. Thus, debris that moves away from the target area immediately distally downstream of the target area can be trapped by the filter-type distal protection device 57, and debris that moves in an opposite direction can be trapped by filter 50. Thus, all downstream locations are filtered and better protected from embolization or occurrence of distal embolisms. It is contemplated that the filter-type distal protection device 57 can be connected and deployed from a guide wire that extends from the inner sheath 30. The filter-type distal protection device 57 can also be placed by a separate incision and placement procedure. In addition, the distal protection device can be formed as a balloon-type distal protection device in which a balloon or balloons can be inflated immediately distal of the target area of the vessel with respect to the filter 50. The balloon(s) can prevent any debris from moving distal of the target area. Other known protection devices can also be used distal to the target area without departing from the spirit and scope of the invention.

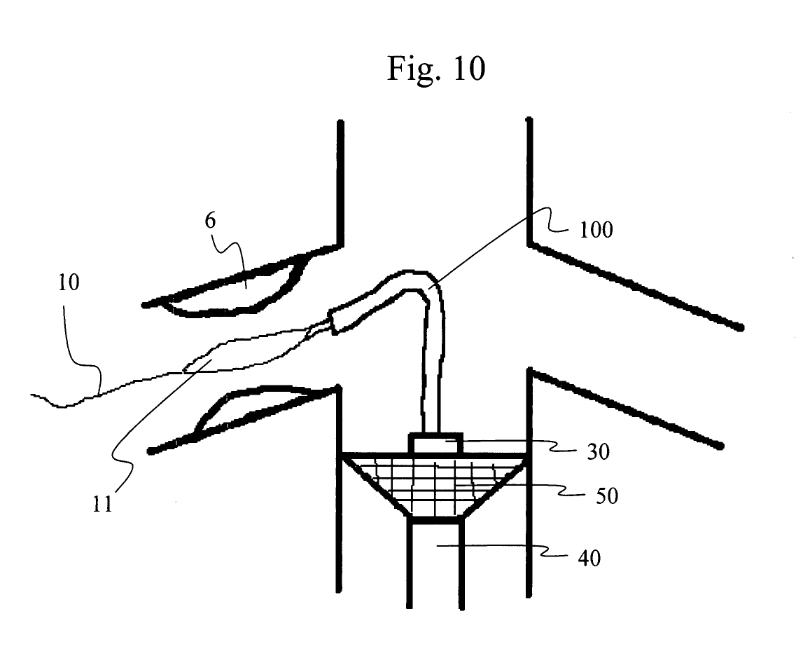

FIG. 10 shows another embodiment of the guiding sheath system in which the inner sheath 30 is formed as a short tip. In this embodiment, a catheter 100 can be extended from the inner sheath 30. The catheter 100 can then be used to guide and locate the interventional device(s) adjacent a lesion 6 in a patient for performance of an interventional procedure.

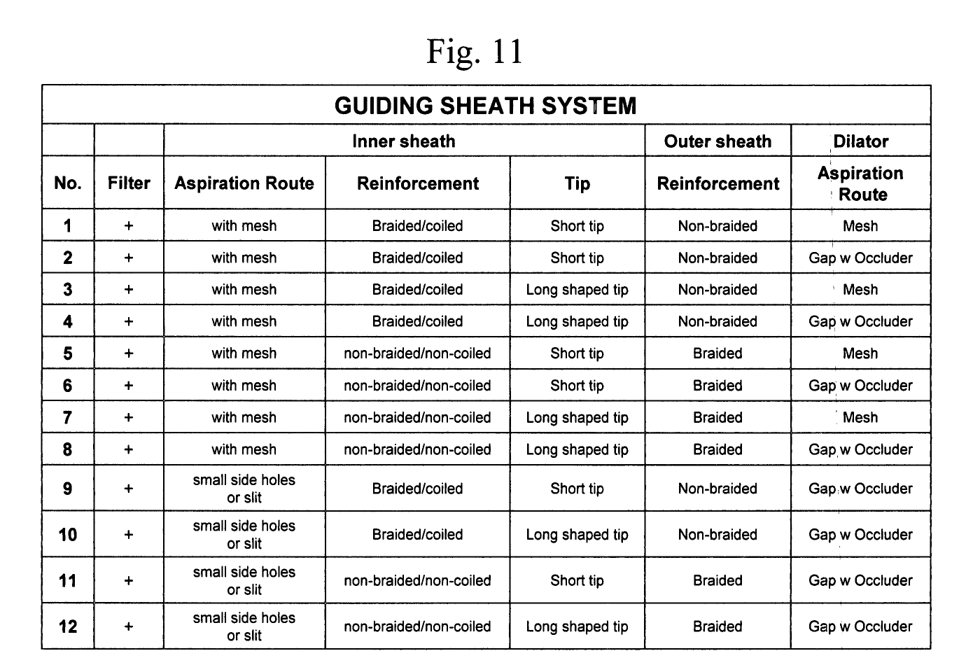

FIG. 11 is a table that shows some of the many variations, combinations and iterations of different structures that are contemplated for use in the guiding sheath system. For example, Sample No. 1 includes an inner sheath 30 that has a mesh portion 31, a short tip (see FIG. 10, for example) and is reinforced by braided or coiled material, such as stainless steel reinforcements 41. The outer sheath 40 of Sample No. 1 is not reinforced by a braided or coiled material. The dilator 20 of Sample No. 1 includes a mesh portion 21 that allows for any debris 5 to be either aspirated or trapped and removed by the guiding sheath system. Each of the samples in the table of FIG. 11 includes a filter 50.

The guiding sheath system of Sample No. 2 includes an inner sheath 30 that has a mesh portion 31, a short tip, and is reinforced by braided or coiled material such as stainless steel reinforcements 41. The outer sheath 40 of Sample No. 1 is not reinforced by a braided or coiled material. The dilator 20 of Sample No. 2 includes a dilator tip occluder 24 that forms a gap for any debris 5 to be either aspirated from or trapped between the inner sheath 30 and dilator 20 and removed by the guiding sheath system.

The guiding sheath system of Sample No. 3 includes an inner sheath 30 that has a mesh portion 31 and a long tip (see FIG. 8, for example) that is either shaped or non-shaped. The inner sheath 30 is reinforced by braided or coiled material, such as stainless steel reinforcements 41. The outer sheath 40 of Sample No. 3 is not reinforced by a braided or coiled material. The dilator 20 of Sample No. 3 includes a mesh portion 21 that allows for any debris 5 to be either aspirated or trapped and removed by the guiding sheath system.

The guiding sheath system of Sample No. 4 includes an inner sheath 30 that has a mesh portion 31, and a long tip that is either shaped or non-shaped. The inner sheath 30 is reinforced by braided or coiled material, such as stainless steel reinforcements 41. The outer sheath 40 of Sample No. 4 is not reinforced by a braided or coiled material. The dilator 20 of Sample No. 4 includes a dilator tip occluder 24 that forms a gap between the inner sheath 30 and dilator 20 for any debris 5 to be either aspirated from or trapped and removed by the guiding sheath system.

The guiding sheath system of Sample No. 5 includes an inner sheath 30 that has a mesh portion 31, a short tip, and is not reinforced by braided or coiled material. The outer sheath 40 of Sample No. 5 is reinforced by a braided or coiled material, such as stainless steel or the like. The dilator 20 of Sample No. 5 includes a mesh portion 21 that allows for any debris 5 to be either aspirated or trapped and removed by the guiding sheath system.

The guiding sheath system of Sample No. 6 includes an inner sheath 30 that has a mesh portion 31, a short tip, and is not reinforced by braided or coiled material. The outer sheath 40 of Sample No. 6 is reinforced by a braided or coiled material, such as stainless steel or the like. The dilator 20 of Sample No. 6 includes a dilator tip occluder 24 that forms a gap between the inner sheath 30 and dilator 20 for any debris 5 to be either aspirated from or trapped and removed by the guiding sheath system.

The guiding sheath system of Sample No. 7 includes an inner sheath 30 that has a mesh portion 31, a long tip, and is not reinforced by braided or coiled material. The outer sheath 40 of Sample No. 7 is reinforced by a braided or coiled material, such as stainless steel or the like. The dilator 20 of Sample No. 7 includes a mesh portion 21 that allows for any debris 5 to be either aspirated or trapped and removed by the guiding sheath system.

The guiding sheath system of Sample No. 8 includes an inner sheath 30 that has a mesh portion 31, a long tip, and is not reinforced by braided or coiled material. The outer sheath 40 of Sample No. 8 is reinforced by a braided or coiled material, such as stainless steel or the like. The dilator 20 of Sample No. 8 includes a dilator tip occluder 24 that forms a gap between the inner sheath 30 and dilator 20 for any debris 5 to be either aspirated from or trapped and removed by the guiding sheath system.

The guiding sheath system of Sample No. 9 includes an inner sheath 30 that has a portion that includes small holes, slits or apertures for aspiration or trapping of debris 5. The inner sheath 30 also includes a short tip and is reinforced by braided or coiled material, such as stainless steel reinforcements 41. The outer sheath 40 of Sample No. 9 is not reinforced. The dilator 20 of Sample No. 9 includes a dilator tip occluder 24 that forms a gap between the inner sheath 30 and dilator 20 for any debris 5 to be either aspirated from or trapped and removed by the guiding sheath system.

The guiding sheath system of Sample No. 10 includes an inner sheath 30 that has a portion that includes small holes, slits or apertures for aspiration or trapping of debris 5. The inner sheath 30 also includes a long tip that is either shaped or non-shaped, and is reinforced by braided or coiled material, such as stainless steel reinforcements 41. The outer sheath 40 of Sample No. 10 is not reinforced. The dilator 20 of Sample No. 10 includes a dilator tip occluder 24 that forms a gap between the inner sheath 30 and dilator 20 for any debris 5 to be either aspirated from or trapped and removed by the guiding sheath system.

The guiding sheath system of Sample No. 11 includes an inner sheath 30 that has a portion that includes small holes, slits or apertures for aspiration or trapping of debris 5. The inner sheath 30 also includes a short tip and is not reinforced. The outer sheath 40 of Sample No. 11 is reinforced by braided or coiled material, such as stainless steel. The dilator 20 of Sample No. 11 includes a dilator tip occluder 24 that forms a gap between the inner sheath 30 and dilator 20 for any debris 5 to be either aspirated from or trapped and removed by the guiding sheath system.

The guiding sheath system of Sample No. 12 includes an inner sheath 30 that has a portion that includes small holes, slits or apertures for aspiration or trapping of debris 5. The inner sheath 30 also includes a long tip that is shaped or non-shaped and is not reinforced. The outer sheath 40 of Sample No. 12 is reinforced by braided or coiled material, such as stainless steel. The dilator 20 of Sample No. 12 includes a dilator tip occluder 24 that forms a gap between the inner sheath 30 and dilator 20 for any debris 5 to be either aspirated from or trapped and removed by the guiding sheath system.

The above described Samples are only exemplary of the types of combinations and iterations of the various structures that can be used in a guiding sheath system made in accordance with the principles of the invention. It should be understood that other various combinations of these structures, materials and relationships between structures can be used for a particular interventional procedure or for a particular patient anatomy, etc. without departing from the scope of the invention.

The materials used for the different components of the guiding sheath system can be those that are commonly used in interventional devices. For example, the inner sheath 30 and outer sheath 40 can be made from PVC, nylon, silicone, polyester, polyimide, polyurethane, polyethylene, polytetrafluoroethylene (PTFE), polymer alloys of these material, and other surgical grade materials and combinations thereof The reinforcements 41 are preferably made from surgical grade stainless steel, but can also be constructed from other materials such as hard plastics, nylon, rigid fabrics, other surgical grade metals and alloys, shape memory alloys, etc. In addition, portions of the inner sheath 30 and outer sheath 40 can be made from different materials such as shape memory alloys or other shaping materials.

The filter 50 can be made from a superelastic alloy such as nitinol (NiTi), surgical grade stainless steel, plastic material that can provide variable stiffness, PVC, nylon, silicone or other surgical grade materials, metals or alloys, including shape memory alloys, and other materials commonly used in vascular filters. Further, the self-expanding feature of the filter 50 can be accomplished in various ways, including through the use of shape memory alloys (e.g., nitinol) incorporated in the filter structure, through the use of inflatable/deflatable portions, through the use of mechanical opening structures, and through the use of other known materials and ways for causing self-expansion of the filter 50. With respect to the inflatable/deflatable portions, it is contemplated that a balloon or balloons can be placed around the periphery of an outer edge of the filter 50 such that when the balloon is inflated, the outer edge of the filter 50 would expand until it securely contacts a vessel wall. Other balloons can be placed along the length of the filter, as may be necessary to ensure full extension of the filter 50 into the vessel and full expansion of the filter 50 to the vessel wall. After the medical procedure is completed, the balloon(s) can be deflated to allow the filter 50 to be retracted back into the outer sheath 40.

The filter 50 can be constructed as a mesh type structure or can be constructed as a thin membrane structure, a structure with various slits or holes therein, a sponge-type structure, or other known structures for use in vascular filters.

The filter 50 can be connected to the inner sheath 30 through the use of bonding methods including welding, use of surgical grade adhesives, and other known bonding methods for surgical devices. In addition, the filter 50 can be connected via other structures to the inner sheath 30 such that the filter 50 is adjacent the inner sheath 30 during operation. For example, the filter 50 can be attached to a separate sheath that is adjacent the inner sheath 30. It is contemplated that the separate sheath would be a small tube shaped structure that would frictionally engage the inner sheath 30 to secure the separate sheath and filter 50 in place within the guiding sheath system. The filter 50 can also be attached via mechanical or suture type coupling elements to the inner sheath 30. The filter 50 can also be attached by integral slots, screw threads or other locking structures that serve to attach the filter 50 to the inner sheath 30. Furthermore, the filter 50 can be located adjacent to the inner sheath 30 without any formal attachment between the two structures.

The dilator 20 can be made from the same various materials from which the inner sheath 30 and outer sheath 40 are made. The shape of the dilator 20 can also vary from the embodiments disclosed above. For example, the dilator indent 22 can include ridges, adhesive, a series of indents/holes, hooks or other structures on an inner surface that help either lock debris to the dilator 20 or facilitate aspiration of the debris from the guiding sheath system. It is contemplated that these structures (ridges, adhesives, a series of indents/holes, hooks or other structures) placed within the dilator indent 22 could help keep debris that moves from the filter 50 through the inner sheath 30 and into the dilator indent 22 from flowing back through the inner sheath 30 or from moving in general. For example, an adhesive located in the dilator indent 22 could cause debris to adhere to the indent wall. Thus, it may be possible to further secure the debris for facilitating debris removal after a medical procedure is finished.

The shape of the dilator tip 23, inner sheath tip 33, and outer sheath tip 43 as shown in FIG. 2 is tapered and preferably atraumatic. However, it should be understood that the tips can be differently shaped and remain in accordance with the principles of the invention. Moreover, the tips can be rounded, hook shaped, or include some sort of ring or bevel at its leading edge. The shape of the tips can vary in accordance with medical personnel's preferences and the patient's needs.

The inner sheath 30 can be formed with a lumen and include means for allowing debris to be removed from a portion of the guiding sheath that includes a mesh portion, a small holes portion, a slit portion, a membrane portion, an elastic storage portion, an indent portion or other portion for storing debris in or allowing debris to pass through the inner sheath 30. The inner sheath 30 can also be formed with ribs or other structures on an interior or exterior surface to promote sliding and/or controlling motion of debris.

With respect to the hub structures and configurations that can be used in accordance with the principles of the invention, the above described configurations are only exemplary. The spacer 80 can be configured in different shapes and materials, and can be located in different positions along the length of the apparatus. In addition, the relative movement between the dilator 20, inner sheath 30 and outer sheath 40 can be different from that disclosed with respect to the embodiments described above. In particular, during an interventional procedure, the inner sheath 30 and dilator 20 can be moved outward to extend from the outer sheath 40 and thus cause the filter 50 to expand into the vessel of a patient. When the spacer 80 is located between the outer sheath hub 60 and Tuohy-Borst valve 70, both the Tuohy-Borst valve 70 and dilator hub 90 can be moved forward towards the outer sheath hub 60 to cause the inner sheath 30 to protrude from the outer sheath 40 and the filter 50 to expand.

The inner sheath 30 can also be retracted into the outer sheath 40 or the outer sheath 40 can be slid forward to envelope the inner sheath 30 and filter 50 at the end of an interventional procedure. These relative motions can be determined by the configuration and relative placement of the outer sheath hub 60, Tuohy-Borst valve 70, spacer 80, and dilator hub 90.

The term interventional procedure refers to both surgical and non-surgical procedures that are performed on the vascular system. For example, interventional procedures include angioplasty, stenting and artherectomy procedures as well as many other types of vascular surgeries and procedures. Further, an interventional procedure can include observatory type procedures in which a surgeon or other medical personnel view portions of the vascular system via a catheterization. Interventional devices can include any medical/surgical device or instrument typically used in interventional procedures.

The type of disease, abnormality or injury that the guiding sheath system is designed to be used for is not limited to those described above in relation to the preferred embodiments of the invention. Moreover, the guiding sheath system can be used in interventional procedures throughout the vascular system. The guiding sheath system can be used to remove plaque or other growth or abnormality from the vascular system, or to repair injuries or congenital defects in the vascular system. As discussed above with respect to FIG. 1, the guiding sheath system can be designed for use in any vessel of the vascular system, including, but not limited to, the renal arteries as set forth by example above.

While illustrative and presently preferred embodiments of the present invention have been described in detail herein, it is to be understood that the inventive concepts may be otherwise variously embodied and employed without departing from the spirit and scope of the invention. The appended claims are intended to be construed to include such variations and equivalents.

What is claimed is:

1. A guiding sheath system for use in an interventional procedure on a patient, comprising:
an inner sheath configured to guide an interventional device to a region of interest in the patient;
an outer sheath configured to be slidable along the inner sheath; and
a filter located adjacent the inner sheath and configured to be expandable.

2. The guiding sheath system according to claim 1, wherein the inner sheath includes a mesh portion.

3. The guiding sheath system according to claim 1, wherein the inner sheath includes a plurality of small holes.

4. The guiding sheath system according to claim 2, further comprising:
a dilator configured to slide in the inner sheath, the dilator including an indent that forms a gap between the dilator and inner sheath.

5. The guiding sheath system according to claim 3, further comprising:
a dilator configured to slide in the inner sheath, the dilator including an indent that forms a gap between the dilator and inner sheath.

6. The guiding sheath system according to claim 1, further comprising:
a dilator configured to slide in the inner sheath, the dilator including a mesh portion.

7. The guiding sheath system according to claim 1, further comprising:
a dilator located adjacent the inner sheath, the dilator including a plurality of holes.

8. The guiding sheath system according to claim 1, further comprising:
a dilator located adjacent the inner sheath, wherein the inner sheath includes a mesh portion and the dilator includes a plurality of holes located adjacent the mesh portion when the dilator is inserted in the inner sheath.

9. The guiding sheath system according to claim 1, wherein the inner sheath includes a shaped tip.

10. The guiding sheath system according to claim 1, further comprising:
a radiopaque marker located adjacent one of the inner sheath and outer sheath such that the position of one of the inner sheath and the outer sheath can be determined during an interventional procedure.

11. The guiding sheath system according to claim 1, wherein the inner sheath includes means for allowing debris to be removed from a portion of the guiding sheath system.

12. The guiding sheath system according to claim 1, further comprising:
a dilator configured to slide in the inner sheath, the dilator including means for removing debris from a portion of the guiding sheath system.

13. The guiding sheath system according to claim 1, further comprising:
a catheter device configured to slide within the inner sheath.

14. A guiding sheath system for use in an interventional procedure, comprising:
an inner sheath including a debris portion;
an outer sheath located adjacent the inner sheath; and
a filter capable of expanding between a contracted state and an expanded state, the filter being configured such that it can be located adjacent the debris portion of the inner sheath when the filter is in the contracted state during use.

15. The guiding sheath system according to claim 14, wherein the debris portion of the inner sheath is formed as one of a mesh portion, a slit portion, and a plurality of holes portion.

16. The guiding sheath system according to claim 14, further comprising:
a dilator located adjacent the inner sheath, the dilator including one of an indent that forms a gap between the dilator and inner sheath, a plurality of holes portion, a slit portion, and a mesh portion.

17. A method for protecting a patient from embolization resulting from an interventional procedure, comprising:
providing a guiding sheath system that includes an inner sheath, an outer sheath, and a filter located adjacent the inner sheath;
advancing the guiding sheath system to a region of interest in a vessel of the patient;
expanding the filter at a location downstream of the region of interest;
deploying a medical device through the inner sheath;
performing an interventional procedure with the medical device on the patient; and
capturing material dislodged by the interventional procedure with the filter.

18. The method for protecting a patient from embolization resulting from an interventional procedure of claim 17, wherein advancing includes advancing the inner sheath and outer sheath to the region of interest in the patient.

19. The method for protecting a patient from embolization resulting from an interventional procedure of claim 17, wherein expanding the filter includes securing the inner sheath and outer sheath in the center of the vessel such that one of the inner sheath and the medical device can extend from the outer sheath without catching on a wall of the vessel.

20. The method for protecting a patient from embolization resulting from an interventional procedure of claim 17, wherein advancing the guiding sheath system includes advancing the inner sheath, and
expanding the filter includes preventing one of the inner sheath and outer sheath from traveling backwards in the vessel due to reaction forces that occur during the interventional procedure.

21. The method for protecting a patient from embolization resulting from an interventional procedure of claim 17, wherein the medical device is a distal protection device.
